(19) United States
(12) Patent Application Publication
(10) Pub. No.: US 2009/0254092 A1
(43) Pub. Date: Oct. 8, 2009

(54) CUTTING APPARATUS FOR PERFORMING OSTEOTOMY

(76) Inventor: Agusti ALBIOL LLORACH, Castellon (ES)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/416,964

(22) Filed: Apr. 2, 2009

(30) Foreign Application Priority Data
Apr. 3, 2008 (ES) P200800991
Nov. 4, 2008 (ES) P200803207

(51) Int. Cl. A61B 17/58

(52) U.S. Cl. 606/87

(57) ABSTRACT

For performing an osteotomy operation at a selected location on a patient's bone, a cutting apparatus comprising a screw device having a tubular shell with at least one longitudinal slot through said shell. A knife holder is slidable longitudinally in the bore to pivotally support at least one knife for displacement between a closed position and an opened position. In its closed position the knife is contained within the bore, and is pivotable to said opened position to project outwardly through the slot to perform the cutting operation, The knife is displaced longitudinally in the bore by an applicator operable to be telescopically displaced in the bore and operable upon its longitudinal displacement to contact an extended end of said knife and pivot the knife into its opened position through the slot, and thereafter to displace said knife holder toward the opposite end of said tubular shell. A spring is positioned between the knife holder and the opposite end of the bore to provide a bias resisting the longitudinal displacement of said knife holder, and upon retraction of said applicator, to return the knife to its closed position.

### FIELD OF INVENTION

The present invention relates to a cutting apparatus for performing osteotomy that introduces essential characteristics of novelty and significant advantages compared to the currently known and used practices.

Specifically, the invention proposes the development of a cutting apparatus for performing osteotomy using a cannulated screw device that enables the performance of straight, parallel, cuts in different planes and at the angles specified. This guarantees faster and more complete healing of the bone in which the operation is performed. Subsequent removal of the cannulated screw enables more complete return of blood supply to the affected zone.

The invention's field of application is the industrial design and manufacture of medical equipment and devices in general, and of the performance of osteotomies in particular.

### BACKGROUND OF THE INVENTION

The term osteotomy is used by persons skilled in the art to describe an operation in which the surgeon performs one or more cuts in a patient's bone in order to achieve changes in its position. Osteotomy operations can, therefore, be carried out, for example to lengthen or shorten a leg bone, correct curvature or angulations, to correctly place the fragments of a broken bone or even sometimes in hip operations to alter the position of the femur.

Currently, two basic cutting apparatuses are known for performing osteotomies: a first apparatus that uses cutting saws and a second apparatus that uses milling cutters. In the first case, the use of a cutting saw requires open surgery, with the drawback that osteotomies are performed by the effort of the surgeon leading to characteristics of precision that are sometimes less than desired; there is also the drawback that the saw vibrates and heats up and the equipment cost is fairly high.

In the second case, that is osteotomy performed with milling cutter, the operation is often usually made through a small incision, this technique being known as minimally invasive surgery (MIS) or laparoscopic surgery. Logically, this can also be performed using open surgery. Similarly to the first case, the surgery is performed by the effort of the surgeon, with the drawbacks of lack of precision and difficulty in controlling the cutter, together with the unwanted heating in the equipment. This equipment is also expensive.

When an osteotomy operation is performed, the surgeon requires the cutting planes to be very parallel so that the healing takes place as quickly as possible. Also, the surgeon must prevent bone fragments from breaking, both to achieve fast healing and to ensure effective surgical correction. It is also necessary to guarantee a good blood supply to the area of the operation because the arteries feeding the bone are destroyed during surgery. Also, to guarantee the maintenance of the correction desired in the operation, effective fixing with the help of pins or screws is required.

### SUMMARY OF THE INVENTION

Taking into consideration the currently existing requirements in the state of the art and the drawbacks associated with the known apparatus and equipment, the current invention's main purpose is to provide an apparatus through which effective solutions could be provided to overcome such drawbacks. This objective has been fully achieved by the apparatus to be described below, whose main characteristics are set down in the claims.

In essence, the apparatus of the invention can be used in both types of osteotomy described above: open surgery or keyhole surgery. The apparatus of the invention implements the embodiment of a cannulated screw device, i.e. internally hollow, prepared to be introduced into the patient's bone in the position in which the osteotomy is to be performed. Once the screw has been installed in the correct position, a knife holder with a cutting knife is positioned in the the hollow bore of the screw in such as way that on displacing it along the bore inside the screw, the knife is opened, performing the osteotomy precisely in the chosen location. An appropriate selection of various cutting tools with different cutting angles enables the surgeon to select the type of osteotomy that is performed. Also, depending on the position of the knives, the osteotomy operation can be performed in different planes and angles. Additionally, there is a spring device fitted inside the cannulated screw that enables the retraction of the cutting tool once the osteotomy has been performed.

As will be understood, an apparatus made in the way proposed by the invention is a much simpler and more efficient apparatus than those known up to now for this type of operation, enables the surgeon to obtain substantially better results in terms of precision and speed compared to the previous technique and the method is much cheaper than the known apparatus briefly described above.

In a first embodiment of the invention, the knife holder and the hollow bore of the screw both have a circular cross section, and it was found that when the applicator is threaded into the proximal end of the screw, the rotary movement of the applicator tends to cause the knife holder to rotate so that the knife is not precisely registered with the slot in the tubular shell of the screw. In a preferred embodiment, the knife holder and the hollow bore of the screw have complementary non-circular cross sections to prevent rotary movement of the knife holder in the bore, and to maintain the knife in registry with the slot in the tubular shell.

Preferably the non-circular cross sections of the knife holder and the bore provide guide surfaces which assure smooth displacement of the knife holder as it is displaced by the applicator, so that the knife travels along the length of the slot as it perform the desired cutting operation on the bone.

### BRIEF DESCRIPTION OF THE DRAWINGS

These and other characteristics and advantages of the invention will be better understood from a detailed description of the invention, given only for illustration purposes, with reference to the figures that accompany it.

FIG. 1 is an elevation view partially sectioned of an example of a preferred embodiment of the system for performing osteotomy with a single knife in the knife holder shown in the closed non-operational position;

FIG. 2 is a similar view to FIG. 1, with the knife in a protruding opened position;

FIG. 3 is a sectional view of the cutting apparatus with the knife in the open operational position taken on the line A-A′ in FIG. 2;

FIG. 4 is an elevation view of the cutting apparatus shown in FIGS. 1-3 of the previous figures, showing the slot for the protrusion of the knife;

FIG. 5 is an exploded elevation view partially sectioned of the cutting apparatus of FIG. 1;

FIG. 6 is an elevation view, partially sectioned, of one variant of the preferred embodiment of the system, with two parallel knives shown in the open operational position;

FIG. 7 is an upper plan view of the knife holder of FIG. 6 with the knives in the open operational position;

FIG. 8 is a sectional view of the knife holder of FIGS. 6 and 7, sectioned vertically by the plane of one of the knives and with the two knives in non-operational closed positions;

FIGS. 9a, 9b, and 9c are completely schematic views of the components making up a first embodiment of the apparatus with a single knife of the invention;

FIGS. 10a and 10b are completely schematic views of a cutting apparatus with two knives in positions of action and withdrawal respectively; and

FIGS. 11a, 11b and 11c are completely schematic views of the possible positions in which the apparatus with multiple knives can work.

### DETAILED DESCRIPTION OF THE EMBODIMENTS

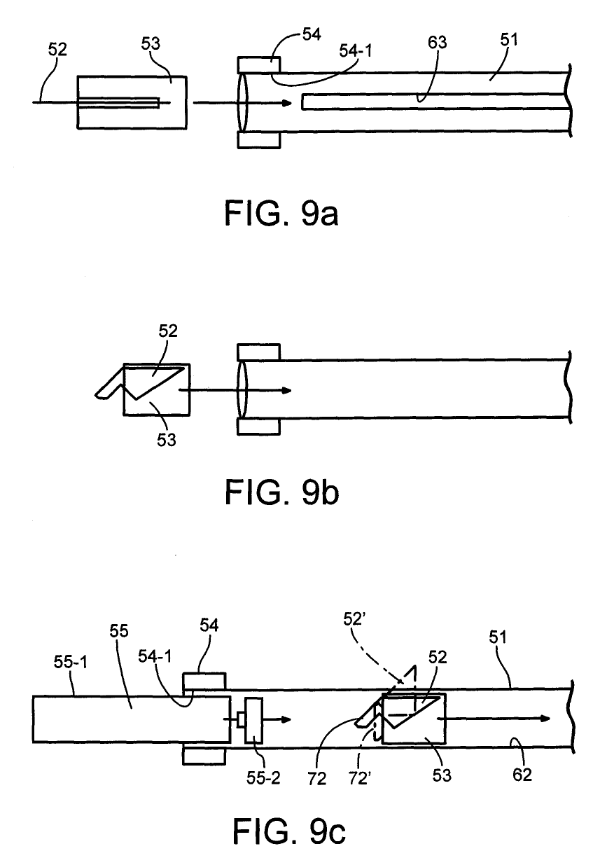

As described above, the detailed description of the embodiments of the apparatus of the invention will follow, with the help of FIGS. 9a through 11c showing schematic views of the components comprising the first embodiments of the cutting apparatus and its operational possibilities. The same components are identified using the same numerical references across the various figures.

Referring in first place to FIGS. 9a, 9b and 9c, the cutting apparatus of the invention comprises a cannulated screw 51 having a tubular shell with external threads (not shown) adapted to be used in an osteotomy operation, with its insertion into the precise point of the patient's bone where it is desired to make the cut. The screw has a hollow interior 62 to allow the passage of one or more cutting knives 52 pivotally mounted in a knife holder 53 for opening and closing pivotal movement. Hereinafter and to simplify the description, reference will be made to a single cutting knife 52, although it should be understood that the description is equally applicable without any change to the case where the knife holder 53 holds more than one knife.

As can be seen in the figures, the tubular shell of the screw 51 has at least one longitudinal slot 63, of a width sufficient to allow the knife 52 pivotally mounted in the knife holder 53 to be projected outwardly to an opened position and to be displaced along the length of the slot 63 as required. The screw 51 also has a grasping portion 54, of greater diameter, at the proximal end thereof. The grasping portion has internal; threads 54-1 adapted to engage external threads 55-1 of an applicator 55.

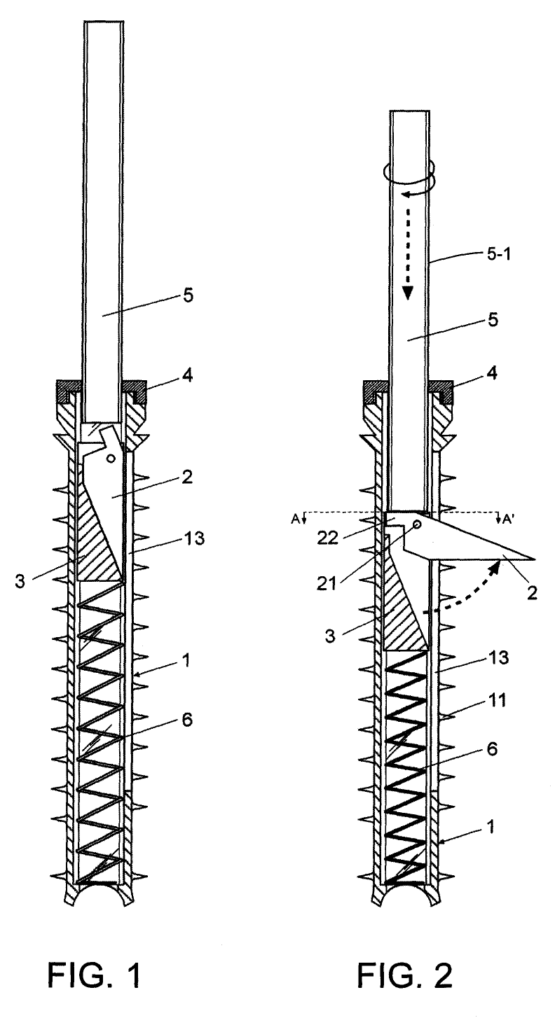

If we observe FIG. 1c, the form of application and action of the apparatus of the invention can be deduced clearly and simply. With the screw 51 positioned in the place where the osteotomy is to be performed, the knife holder 53 with the knife 52 is introduced into the hollow interior bore 62 of the screw. Then the applicator 55 is telescopically introduced into the screw 51, by threaded engagement of its external threads 55-1 with the internal threads 54-4. The applicator 55 advances longitudinally along the hollow interior bore 62 as it rotates, When the front part 55-2 of the applicator reaches the position of the proximal end 72 of the knife 52, its causes the knife to pivot to the opened position, with the consequent emergence of the knife's cutting zone through the longitudinal slot 63. From this time, the cutting apparatus is ready to perform a perfectly straight and guided cut, in the place selected, with absolute precision given the immobility of the screw 51 screwed into the bone. The position of the cut with the knife elevated is shown in broken lines in FIG. 9c with the numerical reference 52′

FIGS. 9a and 9b are completely equivalent, with the only difference that they are rotated by 90° with respect to the longitudinal axis. In the same way, complementarily with the position of the screw 51, the knife holder 53 appears equally in both figures rotated by the same angle. This form of representation graphically illustrates the description just made in the sense that the knife 52 is introduced into the internal space 62 of the screw 51, orientated in such a way that when it pivots, it emerges without difficulty through the slot 63 as has been explained with reference to FIG. 9c.

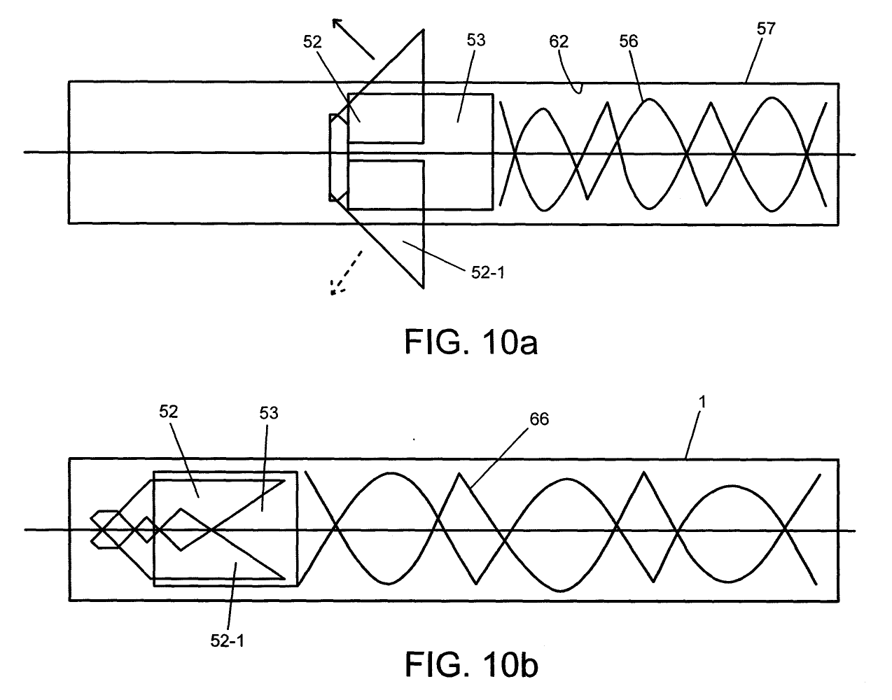

As was previously described, the screw 51 includes therein a spring 56 positioned between the knife holder 53 and the extreme distal end of the hollow interior 62. This situation is clearly visible in FIG. 10a, where the spring 56 presses against the knife holder 53, opposing the displacement of the knife 52 in the longitudinal direction of the screw and helping, therefore, the opening or pivoting of the knife 52 towards its operational position (position of the cut). In the case of FIGS. 10a and 10b, there is a second knife 52-1 shown, associated with the same knife holder 53, in opposition to the first knife 52, and illustrating an alternative embodiment of the invention.

When the cut has been made in the position selected, the spring 66 is an essential element to help extract the knife holder 53 with the knife/knives (52 and 52-1) because the bias of the spring 66 when the surgeon retracts the applicator 55 generates a force on said knife holder 53 in the direction towards the proximal end of the screw, i.e. towards the open end of the screw through which the knife holder 53 was initially introduced. This action is essential for extraction and recovery of the knife holder 53 and of the associated cutting knife/knives 52 and 52-1. The retraction of the applicator permits the knife/knives to assume to the closed position shown in FIG. 10b.

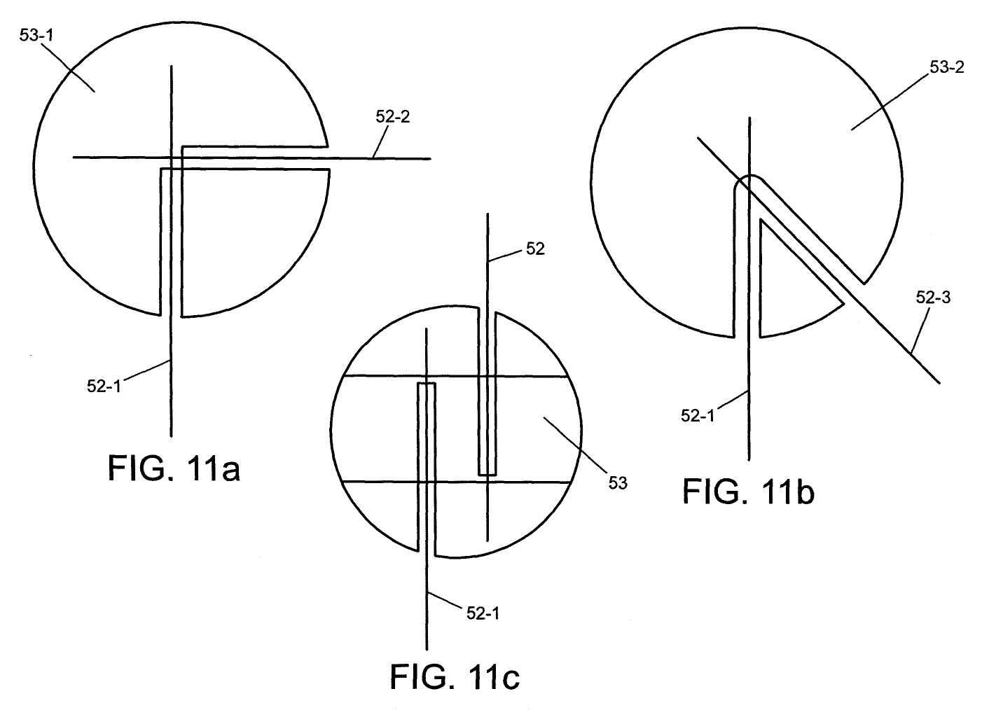

As a complement to what has been described above, FIGS. 11a, 11b and 11c illustrate cutting options that can be achieved with the apparatus of the invention. In FIG. 11a, the knife holder 53-1 includes two knives 52-1 and 52-2 that are fitted in such as way, for example being spaced apart longitudinally of the screw 51, so that they form an angle of 90° to each other. The cut that can be made with the apparatus comprising the knife holder 53.-2 in FIG. 11b is a double cut made by two knives 52-1 and 52-3 at an acute angle to each other, which logically could vary in a wide range of angular magnitudes as required, although in the case illustrated in the example in FIG. 11b is an angle of around 450. In the option illustrated in FIG. 11c, the cut made by the knives 52 and 52-1 in the knife holder 53 are in opposing directions, parallel to each other but in different planes. It is understood that there are many other options that are equally applicable to the apparatus of the invention and are all considered to be included in the invention although they have not been expressly described.

It is not considered necessary to further extend the present description for a person skilled in the art to understand the scope and the advantages derived from it, as well as to carry out a practical embodiment of the object described.

Notwithstanding the above, and given that the description only corresponds to a first embodiment of the invention, it is clear that in essence many variations of detail can be made, all of them protected, that may affect the characteristics of the invention such as the form, size or manufacturing materials of the whole device or of its parts or any other changes that do not alter the essence of the invention.

Additional embodiments of the invention are illustrated in FIGS. 1-5 and FIGS. 6-8 respectively. In the above-described embodiment of the invention, the knife holder and the hollow bore of the screw both have a circular cross section, and it was found that when the applicator is threaded into the proximal end of the screw, the rotary movement of the applicator 55 tends to cause the knife holder 53 to rotate so that the knife is not precisely registered with the slot 63 in the tubular shell of the screw. To minimize this effect, the front part 55-2 of the applicator was designed to minimize the transfer of rotary motion to the knife holder 53. In the preferred embodiments illustrated in FIGS. 1-8, the knife holder and the hollow bore of the screw have complementary non-circular cross sections to prevent rotary movement of the knife holder in the bore. It is noted that in FIG. 9a, the proximal end of the slot 63 is positioned adjacent the grasping portion 54, however the end may be spaced from the grasping portion 54 at the proximal end of the apparatus to provide a closed housing for the knife holder 53 which will maintain the knife it its retracted closed position during the insertion of the cutting apparatus into the selected location on a patient's bone.

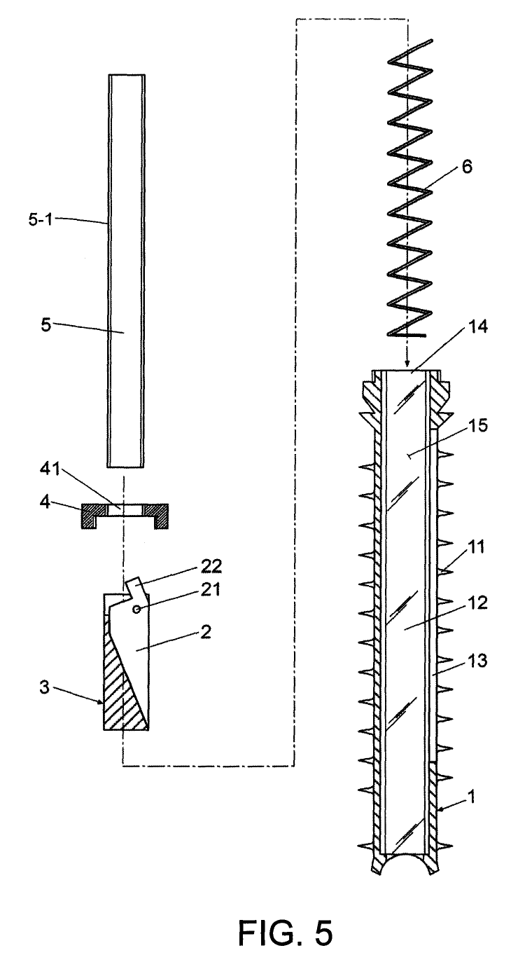

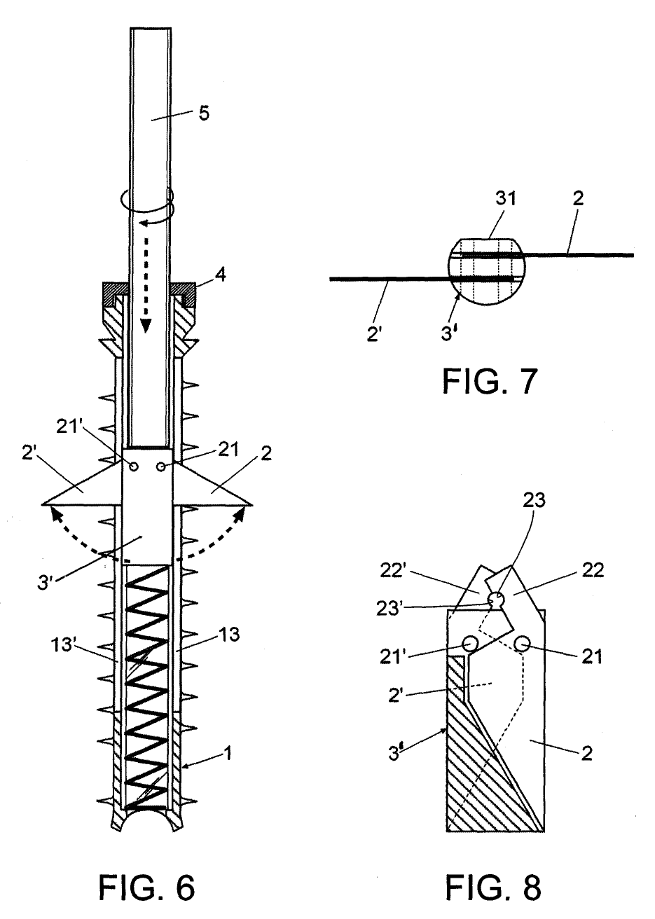

The embodiments of the system for performing osteotomy shown in FIGS. 1-8 comprise a cannulated screw device 1, a knife holder 3, carrying a single knife 2 in FIGS. 1 and 5 and two parallel opposed knives 2, 2′ in FIGS. 6 to 8, an applicator or actuator 5 and a spring 6. The screw device 1 has a tubular shell with an external screw thread 11 for its insertion into the bone to be treated, a hollow interior bore 12 for mounting the spring 6 and the knife holder 3 that can move longitudinally, a longitudinal slot 13, 13′ for each of an the knives 2, 2′ of the knife holder 3 and a rear opening 14 on which an annular detachable collar 4 is mounted, which has a threaded orifice 41 of smaller diameter than the hollow interior 12 of the tubular shell. The applicator 5 is threaded as indicated at 5-1 so that, by threading it into the orifice 41, the applicator may be advanced to greater or lesser extent into the hollow interior of the cannulated screw device 1.

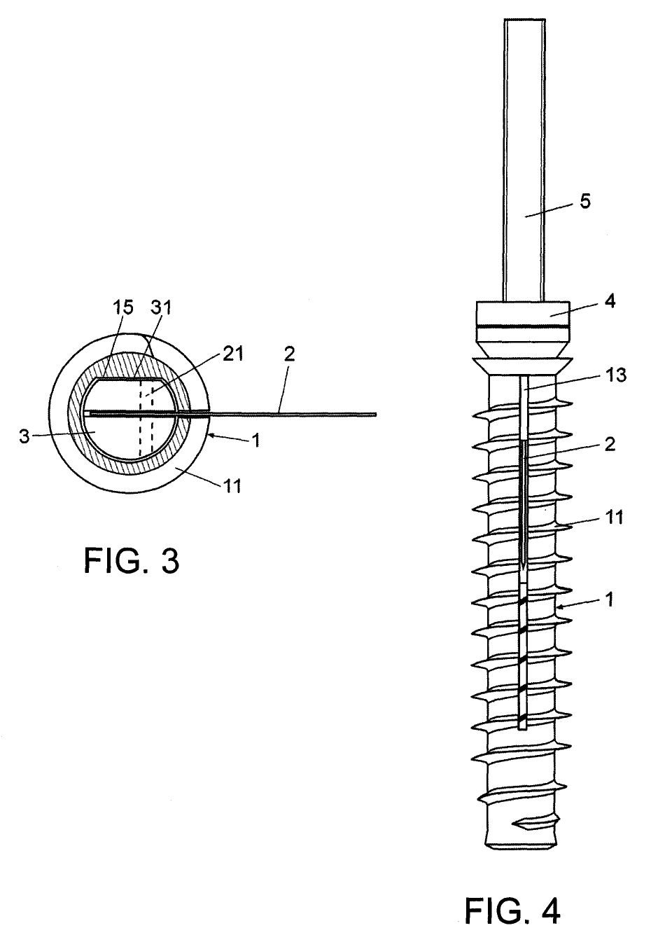

As can be seen clearly in FIG. 3, the knife holder 3 and the hollow bore 12 have a non-circular cross-sections and have complementary longitudinal guides 31, 15 on the lateral opposing surfaces. The guides are shown on both surfaces, flat and parallel, mutually opposed and longitudinally orientated, enabling the longitudinal movement of the knife holder along the hollow bore 12 but prevent its rotation inside said hollow bore 12.

The flat surfaces 31, 15 referred to above are orientated in such as way that the knife holder 3 can only be mounted in a specific angular orientation, in which each knife 2, 2′ registers with the respective longitudinal slot 13, 13′.

Each knife 2, 2′ is mounted in the knife holder on a pivotal axis of folding 21, 21″ that enables its rotation between a non-operational closed position, shown in FIGS. 1 and 7, and an operational opened position, shown in FIGS. 2 and 6 in which it protrudes laterally from the screw device 1 through the corresponding slot 13, 13′.

The proximal end of each knife 2, 2′ has a heel 22, 22′ that emerges from the rear end of the knife carrier 3 with a specific inclination when the knife 2, 2′ is in the non-operational closed position,

The applicator 5, externally threaded as indicated at 5′, is mounted in the threaded orifice (41) of the annular component (4) and can be introduced to greater or lesser extent into the hollow interior bore 12 of the screw device 1. As can be seen in FIGS. 1 and 2, when the applicator starts to be introduced, by threading, into the hollow interior 12 of the screw device 1, it initially acts against the heel 22 of the knife 2 causing it to pivot around the axis 21 towards an operational opened position, shown in FIGS. 2 and 3, in which the knife protrudes through the slot 13. As the applicator 5 is introduced further into the hollow interior bore 12, it causes a longitudinal advance of the knife holder 3, maintaining the knife in an operational position, to perform a straight cut in the patient's bone. This operation occurs in the same way when the knife holder 3 has two knives 2, 2′ and the screw device has two slots 13, 13′, as can be seen in FIG. 6.

The spring 6 which is mounted in the hollow interior bore, in front of the knife holder 3, causes the withdrawal of said knife holder 3 as the applicator 5 is removed from the hollow interior of the screw device. As with the embodiment shown in FIG. 9a, the proximal ends of the slot 13 or slots 13, 13′ may be spaced from the proximal end of the tubular shell to provide a closed housing for the knife holder 3, 3′ when the knives 2,2′ are in their closed positions.

It should be noted that when the knife holder 3′ pivotally mounts two knives 2, 2′, as shown in FIGS. 6-8, each knife has its own pivotal axis 21, 21′, as deter mined by the two pivot pins in the holder 3. As shown in FIG. 8, the proximal ends 22, 22′ of each knife has a cut out 23,23′ adapted to surround the pivot pin of the other knife when the knives are retracted into their closed positions.

Having sufficiently described the nature of the invention and an example of the preferred embodiments, it is hereby declared that the materials, form, size and arrangement of the components described can all be modified, provided that this does not change the essential characteristics of the invention that are claimed below.

1. For performing an osteotomy operation at a selected location on a patient's bone, a cutting apparatus comprising
a screw device having a tubular shell with external threads, a hollow internal bore, and at least one longitudinal slot through said shell, said device adapted to be fixed adjacent said selected location,
a knife holder slidable longitudinally in said bore and having a proximal and a distal end,
at least one knife having a cutting edge at the distal end and an extended end at the proximal end, aid knife being pivotally mounted in said knife holder for displacement between a retracted position and an opened position, said knife in its retracted position dimensioned to be contained within said bore, and being pivotable to said opened position to project outwardly through said at least one slot, in said opened position said cutting edge being transverse to the longitudinal axis of said tubular shell, said extended end projecting beyond the proximal end of said knife holder in the retracted position of said knife,
an applicator operable to be telescopically displaced in the proximal end of said bore and operable upon its longitudinal displacement to contact the extended end of said knife and pivot said knife into its opened position through said at least one longitudinal slot, and thereafter to displace said knife holder toward the distal end of said tubular shell, and
bias means within said distal end of said tubular shell operable to provide a bias resisting said longitudinal displacement of said knife holder, said bias means operable upon retraction of said applicator, to displace the knife holder toward said proximal end and return said knife to its retracted position.

2. A cutting apparatus according to claim 1 wherein said tubular shell has two longitudinal slots therein, and said knife holder has two knives, each adapted to be displaced through said slots by said applicator.

3. A cutting apparatus according to claim 2 wherein said slots and said knives are spaced apart by an angle of 90° about the longitudinal axis of said tubular shell.

4. A cutting apparatus according to claim 2 wherein said slots and said knives are spaced apart by an acute angle about the longitudinal axis of said tubular shell.

5. A cutting apparatus according to claim 2 wherein said slots and said knives are opposite sides of said tubular shell, the cutting edges of said knives being parallel to each other.

6. A cutting apparatus according to claim 2 wherein said knives are spaced apart in said knife holder, whereby the cutting edges of said knives are at different cutting levels.

7. A cutting apparatus according to claim 2, wherein each of said knives has a separate pivotal axis, and is mounted for pivotal movement on a separate pivot pin in said holder, the extended end of each knife having a cutout surrounding the pivot pin of the other knife.

9. A cutting apparatus according to claim 1 wherein said bias means comprises a spring mounted in the distal end of said tubular shell.

10. A cutting apparatus according to claim 1 wherein said slot has a proximal end spaced from the proximal end of said tubular shell to provide a closed housing for said knife holder which encloses the knife holder and maintains the knife it its retracted position within the tubular shell.

11. A cutting apparatus according to claim 1 wherein said applicator is threaded and said tubular shell has a threaded aperture at its proximal end to engage the threads of said applicator and effect longitudinal displacement of the applicator upon rotation of the applicator about said longitudinal axis of the tubular shell.

12. A cutting apparatus according to claim 11 wherein the bore of said tubular shell has a non-circular cross section, and said knife holder has a complementary cross section, whereby the knife holder is prevented from rotating about the longitudinal axis of said shell when said applicator is rotated.

13. A cutting apparatus according to claim 12, wherein said bore has a first flat surface extending parallel to the longitudinal axis of said tubular shell along its full length, and said knife holder has a second complementary flat surface adapted to slide along said first flat surface.
